(19) United States
(12) Patent Application Publication
(10) Pub. No.: US 2022/0054674 A1
(43) Pub. Date: Feb. 24, 2022

(54) PORTABLE MODULAR SANITIZER FOR THE DISINFECTION OF PRODUCTS FOR PROMPT DELIVERY

(71) Applicant: GADOTTI CAR SERRALHERIA LTDA-ME, Extrema (BR)

(72) Inventor: Eduardo Gadotti Martins, Extrema (BR)

(73) Assignee: GADOTTI CAR SERRALHERIA LTDA-ME, Extrema (BR)

(21) Appl. No.: 17/110,812

(22) Filed: Dec. 3, 2020

(30) Foreign Application Priority Data
Aug. 20, 2020 (BR) BR1020200169610

(51) Int. Cl. A61L 2/10; A61L 2/20

(52) U.S. Cl. CPC A61L 2/10; A61L 2/202; A61L 2202/16; A61L 2202/20; A61L 2202/121; A61L 2202/15; A61L 2202/11

(57) ABSTRACT

Portable modular sanitizer (10) for disinfecting various products (Pd), like food packages for delivery, goods in general as commercialized in supermarkets, drugstores, convenience stores or similar commercial establishments, as well as letters, objects, packages or any other inanimate product. The portable modular sanitizer (10) being presented in the form of a box (11), cabin (12) and backpack (13), all of them by a timer (TP) to control the time of incidence of light spectra (21) emitted by UVC lamps (20) installed inside the receptacle (11A), (12A) or (13A) constituting the sanitizer (10) which UV-C ultraviolet light (21) germicide effect is the result of a photolytic effect eliminating the RNA and DNA replication potential, inactivating and eliminating the COVID-19 virus, as well as other virus and bacteria existing on the product (Pd) surfaces.

### FIELD OF THE INVENTION

The present patent of invention refers to a portable modular sanitizer for disinfecting products for prompt delivery like food packages, merchandises in general commercialized by supermarkets, drugstores, convenience stores or similar commercial establishments, as well as letters, objects, packages or any other inanimate product. Said portable modular sanitizer may be presented as a box with a hinged lid, cabin or backpack for food transport delivery, all provided with temporizing means for one or more germicide UV-C lamps, linked or not to the application of ozone gas for adequately disinfecting stored products by means of the light spectrum, thus providing the elimination of between 90% and 100% virus and bacteria, including SARS-Cov-2, COVID-19, by destroying the genetic material of the viral and bacterial DNA or RNA.

### BACKGROUND OF THE INVENTION

It is known that the pandemics called the attention to various issues, such as e.g. the contamination by direct or indirect contact through surfaces or objects contaminated by virus such as the one causing COVID-19 and/or others, such as bacteria, caused when infected persons, by means of secretions such as saliva and respiratory secretions or his/her respiratory droplets, which are expelled when someone coughs, sneezes or speaks, contact the surface of e. g. products exposed on the shelves of supermarkets, drugstores, convenience stores or similar commercial establishments.

Generally speaking, the virus SARS-Cov2, which causes COVID-19, after contacting a surface, and depending on the characteristics of that surface, may remain active for a few hours or even days, such as e. g. 72 hours on plastics and stainless steel, 24 hours on carton and four hours on copper, among others.

However, we have noticed during this long quarantine period a substantial increase in delivery requests, be it for food, but also for medicines, essential goods, clothing and numerous other products, thus considerably increasing the possibility of virus “transit” through the packages. Despite the guidance for package hygiene during the contact and even hand hygiene after the contact, we can state that a large portion of the public does not follow these rules, and, as time goes by, hygiene guidance will much probably be forgotten.

Therefore, we can notice that one of the largest inconveniences found in these times of high contamination is the fact that the exposed or commercialized products in commercial establishments and sold for delivery configure themselves as possible spreading agents for highly contagious diseases, thus requiring disinfection, preferably before they enter home, commercial, service and other environments.

Currently, there are products offering such disinfection, especially aerosol products for spraying over commercialized product surfaces. However, despite offering disinfection, the full reached surface will depend on the applicator's ability, thus promoting ineffective disinfection.

### STATE OF THE ART ANALYSIS

By searching specialized databases, we have found documents presenting sanitizers, such as e. g. document n° BR10.2012.005708-5, referring to equipment for sanitizing personal objects, notably helmets, gloves, tennis, boots and clothing, which are widely used by motorcyclists, cyclists and athletes in a wide range of modalities, said equipment being included in the field of methods or devices for sanitizing materials or objects, being said sanitizing equipment liable for handling by the user him/herself, thus provided with a closed place for positioning the helmet and other objects to be sanitized, a panel controlling the insertion of a sanitizing element for performing sanitization and its respective activation by an inductive, debit/credit or RFID card, being thus liable to be installed in motorcycle re-sellers, motorcycle article and/or sporting article stores, thus providing an important service for maintaining the user's health.

Document n° BR20.2014.006540-2 discloses a surface disinfection device using a UVC lamp to disinfect surfaces, acting for microbiological control, comprising: a UVC lamp, the equipment body, a tension source, a reflector, a reactor, level sensors, a control board, an activating button and a blue LED indicator.

Document n° BR 0005153-5 discloses a system and device for sterilizing environmental air by means of ultraviolet radiation at light speed. Based on the emission of ultraviolet light beams by a compact germicide UVC lamp, so to increase the efficiency of ultraviolet rays and produce activated oxygen and negative ions purifying the breathing air, said germicide UVC lamp is installed in an ultraviolet sterilizing chamber, through which the air in the environment circulates and is there sterilized, also including a set of filters intended to retain bacteria, virus, fungi and other constituents in suspension, a photo-oxidization chamber sterilizing bacteria and virus which cannot be retained by the finer filters, stainless steel air mirrors contributing to model the disinfection area and concentrate the yield of the germicide UVC lamp to increase its germicide effectiveness, advanced electronic controls regulating air quality, eliminating toxic emanations, gases and particles, releasing it from micro-organisms and impurities in suspension, electronic monitors following volume and air flow paths, as well as the moment when the filters and the germicide UVC lamp should be substituted.

Despite being included in the same field of application, the above-mentioned documents are different from the present invention, as we will see below, thus assuring that it fully meets legal patentability requirements.

### OBJECTS OF THE INVENTION

Always looking for social good and making daily tasks become easier, and especially to adequate itself to the new social needs brought by the pandemics, as well as innovating the cargo storage and transportation field with new products intended to the whole population of distributors and delivery men and women, aiming to transport/store and/or sanitize goods in a safe, efficient and healthy way, the applicant has developed a portable modular sanitizer to disinfect objects, goods, merchandises and foods, so to allow them to safely reach their final destination, including the disinfection of constantly used masks which should be substituted every three hours.

Therefore, the object of the invention is to disclose a portable modular sanitizer for sanitizing products for prompt delivery, such as goods in general commercialized by restaurants, snack bars, supermarkets, drugstores, convenience stores or similar commercial establishments, as well as letters, objects, packages or any other inanimate product.

An object of the invention is to disclose a portable modular sanitizer presented in various shapes, such as a box with a hinged lid, cabin or backpack for food transport delivery, all of them provided with temporizing means for germicide UV-C lamp(s) for disinfecting stored products by the action of the light spectrum, thus providing for the elimination of between 90% and 100% virus and bacteria, including SARS-Cov-2, causing COVID-19, by destructing the genetic material of DNA or RNA virus and bacteria.

Another object of the invention is to disclose a portable modular sanitizer with a safety system comprised by a set of highly effective sensors which, when activated by manually opening the door, even if the sanitizing process is immediately interrupted before the external light goes off, promptly turning off the UV light and thus avoiding contact between the illuminated surface and the skin of the operator.

Another object of the invention is to disclose a portable modular sanitizer in the shape of a box with hinged lid, with ideal measurements for delivery services, which object is to sanitize goods from the food, corporate, home, health and tourism fields by means of the incidence of the germicide UV-C light spectrum and, as a complement in a few cases, the ozone gas, thus eliminating up to 99% bacteria and the SARS-Cov-2 virus.

Another object of this invention is to disclose a portable modular sanitizer in the form of a cabin, allowing for disinfection by the association between germicide UVC lamps and ozone destroying the genetic material of virus and bacteria DNA or RNA, also allowing all goods inside a supermarket cart/basket to be disinfected one single time, allowing the products to reach the client's vehicle with up to 99% bacterium elimination, thus avoiding the risk of contracting virus and/or bacteria present on the packages and/or products.

Another object of the present invention is to disclose a portable modular sanitizer in the form of a backpack allowing to disinfect goods packed during a trip, assuring that, between the collection and the delivery to the client, the product is sanitized, eliminating between 90% and 100% virus and bacteria, including SARS-Cov-2.

### BRIEF DESCRIPTION OF THE DRAWINGS

As a complement to the present description, so to reach better understanding of the characteristics of the present invention and following a preferential embodiment, a disclosure of the attached set of drawings follows, representing the following as an example, but with no limitation

FIG. 1 shows a perspective view of the presented embodiments of the sanitizer, showing the means for controlling a germicide UV-C lamp, associated or not to the application of ozone gas for the appropriate temporization of stored product disinfection;

FIG. 2 discloses a perspective view of the sanitizer in the form of a box with hinged lid, showing its respective constituents;

FIG. 2A shows a crosswise cut A.A. as shown by FIG. 2;

FIG. 3 shows a perspective view of the sanitizer in the form of a cabin showing its respective constituents;

FIG. 3a shows a crosswise cut view A.A as shown by FIG. 3;

FIG. 4 shows a perspective view of the sanitizer in the form of a backpack showing the respective constituents; and

FIG. 4A shows a crosswise cut view A.A as shown by FIG. 4.

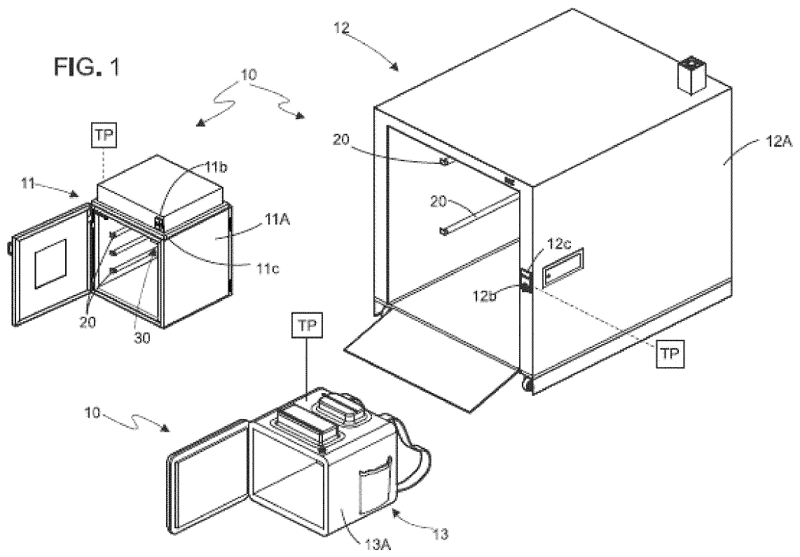

### DETAILED DESCRIPTION OF THE INVENTION

With reference to the shown figures, the present patent of invention refers to a “PORTABLE MODULAR SANITIZER FOR DISINFECTING PRODUCTS FOR PROMPT DELIVERY”, which sanitizer (10) is based on UVC lamps (20) for disinfecting various products (Pd) of the type commercialized in supermarkets, drugstores, convenience stores or similar commercial establishments, as well as letters, objects, packages or any other inanimate product.

According to the present invention, said modular sanitizer (10) is portable, more precisely configured by a box (11), cabin (12) and/or backpack (13), each one provided with a temporizer (TP) to control the time of incidence of light spectrum (21) emitted by the UVC lamps (20) installed inside the receptacle (11A), (12A) or (13A) constituting the sanitizer (10), wherein the germicide effect of the UV-C ultraviolet light (21) is the result of a photolytic effect eliminating DNA and RNA replication potential, inactivating and eliminating the COVID-19 virus, as well as other virus and bacteria existing on the product (Pd) surfaces, and said sanitizer (10) includes a safety system (30) formed by sensors (31), also installed inside the receptacle (11A), (12A) or (13A), immediately interrupting the cycle of incidence of light spectra (21) from the UVC lamps (20) when accessing the inner side of the sanitizer (10), thus avoiding the contact between the illuminated surface and the skin of the operator.

Each sanitizer (10) also contemplates an activating button (11b), (12b) for the sanitization process by the incidence of light spectra (21) from the UVC lamps (20) and an emergency button (11c) and (12c) which may be activated by the operator in case of need to interrupt the sanitization process, besides a lamp or a light emitting diode LED (11d), (12d) and (13d), indicating the operational execution of the sanitization cycle for the products (Pd) packed in the receptacles (11A), (12A) or (13A) and, when turned off, it indicates the end of the cycle.

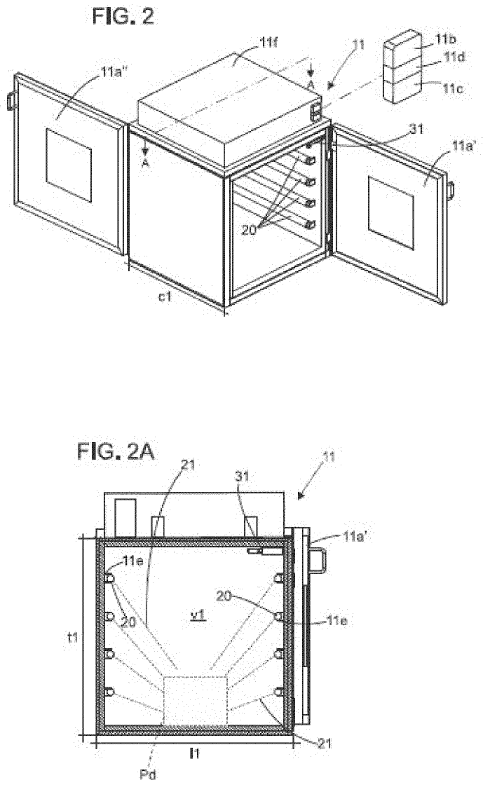

In a first version, the sanitizer (10) (please refer to FIGS. 2 and 2A) is presented in the form of a box (11) comprised by a receptacle (11A) with hinged doors (11a′) and (11a″) and which measurements width (I1), height (t1) and length (c1) constitute ideal volume (v1) and dimensions for delivery services. The internal surfaces of both side walls of the receptacle (11A) receive supports (11e) to install the lamps (20) and also includes a limit stop switch (31) constituting the safety system (30).

The upper face of the receptacle (11A) receives a secondary box (11f) to assemble the battery (not shown) and electronic constituents such as the timer (TP), in which the activating (11b) and emergency (11c) buttons are located, as well as the button indicating the operational execution of the sanitization cycle (11d).

To disinfect the products (Pd) in the box (11), you just need to insert it in the receptacle (11A) and close the hinged doors (11a′) and (11a″), activating it by clicking the button (11b), wherein, as soon as the operational execution indicator (11d) for the sanitization cycle for the packed products (Pd) is off, you can just open the door (11a′) and take off the product (Pd), sanitized up to 99%.

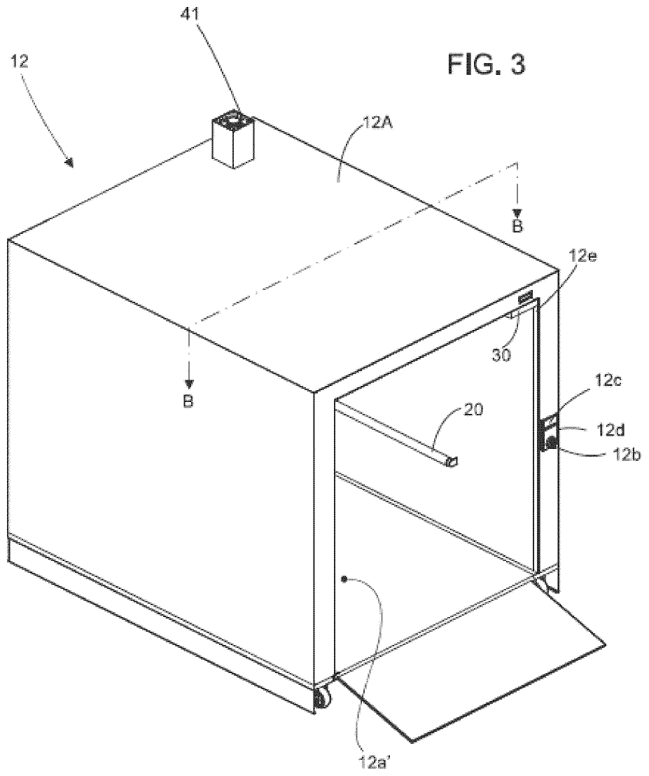

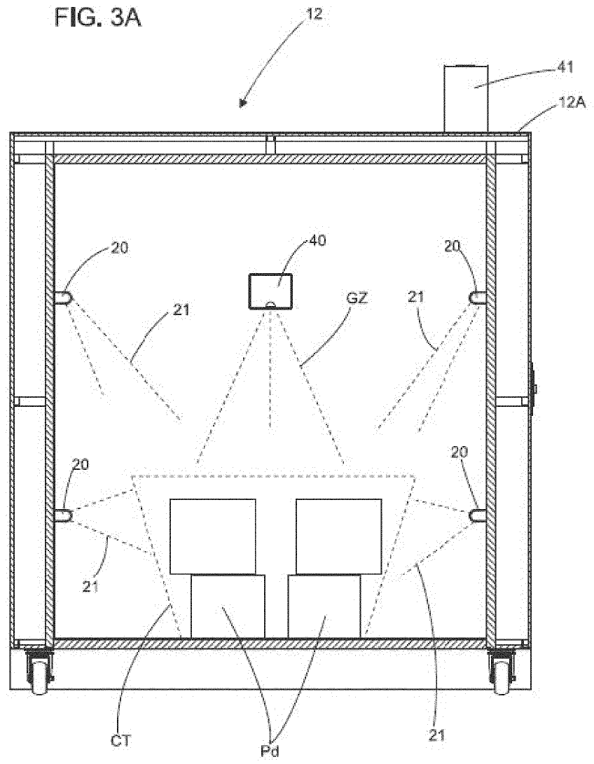

In a second version (please refer to FIGS. 3 and 3A), the sanitizer (10) has the form of a cabin (12) with ideal measurements to receive a supermarket cart/basket (CT) with respective products (Pd) and a receptacle (12A) provided with a front opening (12a′), wherein a folding door (12e) is installed, and the internal surfaces of both side walls of the receptacle (12A) receive the UVC lamps (20), while the internal surface of the end wall receives an ozone gas (Gz) spray (40) and an exhaust fan (41) is installed on the upper wall.

Next to the folding door (12e), the safety system (30) may be installed, and the activating (12b) and emergency (12c) buttons, as well as the button indicating the operational execution of the sanitization cycle (12d), are on the edges of the side walls.

To start the sanitization process in the cabin (12), you should just insert the cart/basket (CT) with the respective products (Pd), close the cabin door (12e) and activate the button (12b), in a process which may last approximately one minute, being 50 seconds to apply the light spectra (21) from the lamp and ozone gas (Gz) to the products (Pd) and 10 final seconds of forced ozone exhaustion to the external side of the sanitizer (10) by means of the exhaust fan (41), thus eliminating any direct contact between the gas and the equipment operator. When the sanitization time is concluded, the display located on the front part of the receptacle (12A) indicates the end of the process, releasing the operator to open the door (12e) to safely take off the cart/basket.

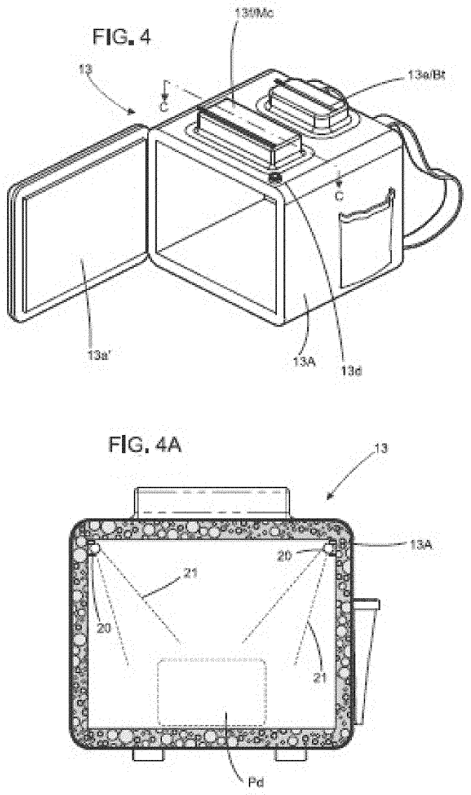

In a third version (please refer to FIGS. 4 and 4A), the sanitizer (10) has the form of a backpack (13) comprised by a receptacle (13A) provided with a hinged door (13a′), wherein lamps (20) are installed on the internal surfaces of its side walls. The upper face of the receptacle (13A) includes a pocket (13e) to assemble a battery (Bt) and a packet (13f) to assemble a card machine (Mc), as well an operational execution indicator (13d) for the sanitization cycle of the packaged products (Pd).

For disinfection, you just need to insert the products (Pd) into the internal portion of the receptacle (13A) and close it, wherein, during the trip, the sanitization process will start and be automatically turned off after the correct sanitization period, delivering the sanitized product (Pd) to the client.

It is known that, when the present invention is put into practice, modifications may be introduced to given embodiment details and form, not escaping from the fundamental principles as clearly substantiated in the claims, being thus understood that the used terminology did not have a limitation purpose.

1. A portable modular sanitizer for disinfecting products for prompt delivery comprising:
a box (11),
a cabin (12);
a backpack (13),
wherein each one of the box, the cabin, and the backpack include with a timer (TP) to control incidence time for light spectra (12) emitted by a UVC lamps (20) installed inside each one of the box, the cabin, and the backpack, a UV-C ultraviolet light (21) germicide effect is the result of a photolytic effect eliminating DNA and RNA replication potential, inactivating and eliminating COVID-19 virus, as well as other virus and bacteria existing on the surfaces of the products (Pd),
a safety system (30) including sensors (31) is installed within the box, the cabin, and the backpack, the safety system (3)) immediately interrupts incidence cycle for light spectra (21) from the UVC lamps (20) during the access to the inner side of the sanitizer (10), avoiding the contact between an illuminated surface and an operator skin;
an activating button (11b), (12b) for the sanitization process by the incidence of light spectra (21) from the UVC lamps (20); and
an emergency button (11c) and (12c) which is activated by an operator in case of need to interrupt the sanitization process, besides a lamp or a light emitting diode LED (11d), (12d) and (13d), indicating the operational execution of the sanitization cycle for the products (Pd) as packed in the receptacles (11 A), (12A) or (13A) and, when turned off, it indicates the end of the cycle.

2. The portable modular sanitizer according to claim 1, wherein the box (11) includes a receptacle (11A) with hinged doors (11a′) and (11a″), which measurements of width (I1), height (t1) and length (c1) constitute ideal volume (v1) and dimensions for delivery services; an internal surfaces of both side walls of the receptacle (11A) include supports (11e) for installing lamps (20), as well as a stop end switch (31) constituting the safety system (30); the upper side of the receptacle (11A) includes a secondary box (11f) to assemble the battery and electronics such as the timer (TP), and the activating (11b) and emergency (11c) buttons, as well as the indicator for the operational execution of the sanitization cycle (11d), are installed therein.

3. The portable modular sanitizer according to claim 1, the cabin (12) is adapted to receive a supermarket cart/basket (CT) with respective products (Pd) and the cabin (12) includes a receptacle (12A) including a front opening (12a′) in which a folding door (12e) is installed, internal surfaces of both side walls of the receptacle (12A) including the UVC lamps (20), while an internal surface of an end wall of the cabin includes an ozone gas (Gz) sprayer (40) and, on an upper wall of the cabin includes an exhaust fan (41); next to the folding door (12e), the safety system (30) is located installed, as well as the activating (12b) and emergency buttons (12c), and the indicator for the operational execution of the sanitization cycle (12d), on the edges of the side walls.

4. The portable modular sanitizer according to claim 3, wherein activation of the sanitization process in the cabin (12) includes inserting the cart/basket (CT) with respective products (Pd), closing of the cabin door (12e) and activation of the button (12b), wherein said process may last approximately 1 minute, being 50 seconds for the application of light spectra (21) from the lamp and ozone gas (Gz) to the products (Pd) and 10 final seconds for the forced ozone exhaustion to the external side of the sanitizer (10) through the exhaust fan (41), thus avoiding any direct contact between the gas and the equipment operator.

5. The portable modular sanitizer according to claim 1, wherein the backpack includes a receptacle (13A) provided with a hinged door (13a′) on which side wall includes internal surfaces lamps (20); on the upper face of the receptacle (13A), a pocket (13e) is included to assemble the battery (Bt) and a pocket (13f) to assemble the card machine (Mc), as well as an indicator for the operational execution (13d) of the sanitization cycle for the packaged products (Pd).
